(19) United States
(12) Patent Application Publication
(10) Pub. No.: US 2003/0036086 A1
(43) Pub. Date: Feb. 20, 2003

(54) Holder for multiple well sequencing/PCR plate

(76) Inventors: Jack Thacher Leonard, South Hamilton, MA (US); Joseph E. Gabriels JR., Arlington, MA (US); Masaharu Mabuchi, Beverly, MA (US); Gary LaBombard, Concord, NH (US)

Correspondence Address: MILLIPORE COPORATION, 80 ASHBY RD, BEDFORD, MA, 01730, US

(21) Appl. No.: 10/219,401

(22) Filed: Aug. 15, 2002

Related U.S. Application Data
(60) Provisional application No. 60/312,708, filed on Aug. 16, 2001.

(51) Int. Cl. C12Q 1/68; B25B 1/20; C12M 1/34

(52) U.S. Cl. 435/6; 435/287.2; 269/45

(57) ABSTRACT

A system for holding a multiple welled plate during processing comprised of a multiple welled plate, typically containing 96, 384 or more individual wells, each having an open bottom closed by a membrane and a holder for such a plate. The holder is formed with a filter support portion that supports the lower surface of the filter during processing. The holder may optionally be thermally conductive or if not thermally conductive, may include an additional thermally conductive layer between the lowermost surface of the filter and the filter support portion to provide for thermal cycling applications.

The present invention relates to a holder for a multiple welled plates. More particularly, it relates to a holder for multiple welled plates used in automated equipment for DNA sequencing and/or PCR reactions.

### BACKGROUND OF THE INVENTION

The need for rapid, high throughput screening of genomic materials has led to the development of automated equipment for the preparation and processing of small volume samples.

Currently, one prepares a number of samples in a multiple welled plate, typically containing 96, 384 or more individual wells, each having an open bottom closed by a membrane. The samples are treated in the wells with various liquids such as dye terminators, buffer solutions, etc. which are removed by centrifugation or a constant pressure differential (negative or positive). The samples are then resuspended in a liquid such as water or a buffer with agitation such as shaking or pipeting the liquid up and down several times. It is then transferred to a solid bottomed plate for further processing, for example in a DNA sequencer. The purified samples are then electrokinetically injected into capillary electrophoresis instruments.

It has been found that a solid bottomed plate is necessary to work with the robotic devices that accompany such devices. This leads to an additional manual step that is time consuming, prone to error and prone to loss of sample. What is desired is a device that would allow one to use the existing filter bottomed multiple welled plate directly in sequencing processing and analysis equipment.

The present invention provides such a device and system.

### SUMMARY AND OBJECTS OF THE INVENTION

The present invention provides a system formed of a multiple welled plate, typically containing 96, 384 or more individual wells, each having an open bottom closed by a membrane and a holder for such a plate. The holder is formed with a filter support portion that supports the lower surface of the filter during processing.

Moreover, the present invention provides a holder per se for multiple welled plates comprising an outer periphery that matches the outer periphery of the plate, a filter support portion inward of the outer periphery and matching the area of filter of the plate and one or attachment devices for releasably securing the plate to the holder in a defined location.

It is an object of the present invention to provide a system formed of a multiple welled plate, typically containing 96, 384 or more individual wells, each having an open bottom closed by a membrane and a holder for such a plate wherein the holder is formed with a filter support portion that supports the lower surface of the filter during processing.

It is another object of the present invention to provide a holder for multiple welled plates comprising an outer periphery that matches the outer periphery of the plate, a filter support portion inward of the outer periphery that substantially matches the area of filter of the plate and one or attachment devices for releasably securing the plate to the holder in a defined location.

It is an additional object of the present invention to provide a system formed of a multiple welled plate, each of the wells having an open bottom closed by a membrane and a holder for such a plate wherein the holder is formed with a filter support portion that supports the lower surface of the filter during processing and contains a means for transfer heat to the plate for thermal cycling. If the samples are to be heated from above or below, the filter by means of heated pins, plate or other device, the system will provide the means to prevent evaporation from the top and bottom of the filter plate.

It is a further object of the present invention to provide a process for direct injection processing comprising the steps of taking a multiple welled filter plate and preparing a series of purified samples in one or more of the wells, attaching the plate to a holder for the plate, the holder having a upper portion corresponding in size and location to the filter area of the plate so as to support the filter and limit or prevent the filter's vertical movement during processing, adding one or more fluids to the samples in the one or more wells of the plate to resuspend the samples, placing the plate/holder into a direct inject processing device and performing a direct inject process.

### IN THE DRAWINGS

FIG. 1 shows the present system in an exploded planar view view.

FIG. 2 shows a top down view of a plate useful in the present invention.

FIG. 3 shows a cross sectional view of one embodiment of the system of the present invention.

FIG. 4 shows a cross sectional view of another embodiment of the system of the present invention.

### DETAILED DESCRIPTION OF THE INVENTION

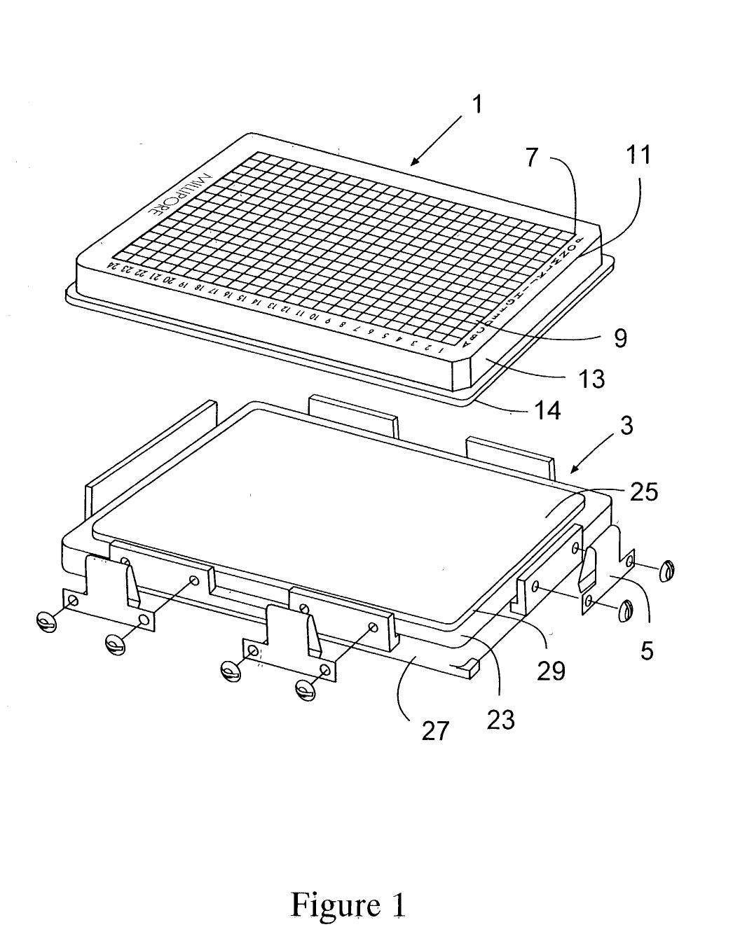

The present invention is a system for holding multiple welled filter plates during processing comprised of a plate and a holder for the plate. FIG. 1 shows a first embodiment of the present invention in exploded planar view.

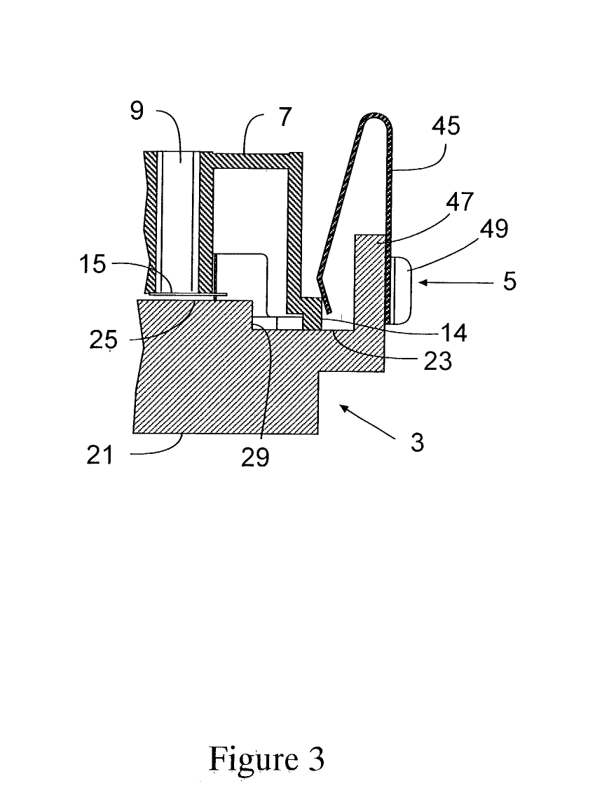

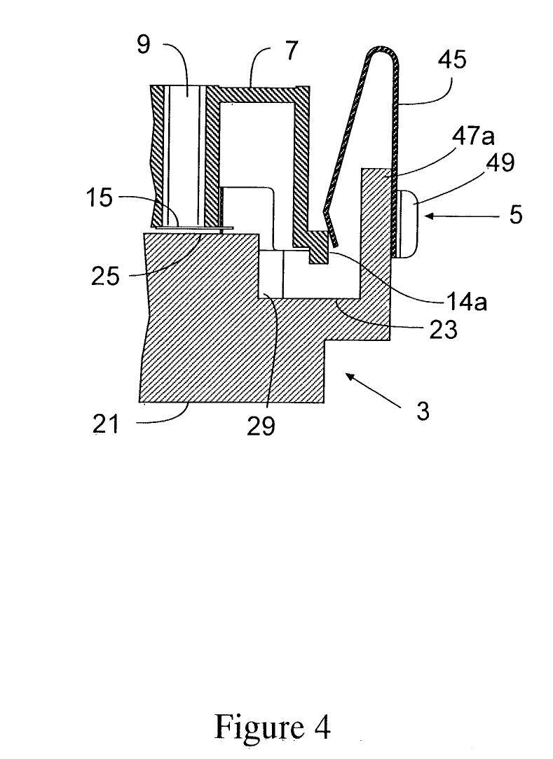

The plate 1 is attached to a holder 3 by one or more attachment devices 5. The plate is a standard multiple welled plate such as a MULTISCREEN™ SEQ plate available from Millipore Corporation of Bedford, Massachusetts. The plate has a top substantially planar surface 7 which contains a series of wells 9 that extend perpendicularly downward from the top surface 7 to a predetermined depth. The outer peripheral edge 11 of the top surface 7 extends beyond the outermost wells 9 of the plate 1 and has a downwardly depending skirt 13 that is slightly greater in vertical length than the wells 9. The bottommost portion of the skirt 13 terminates in a lip 14 as is well known with such multiple welled plates. As shown the lip 14 is relatively small in height. As is well-known in the art, this lip 14 may also be of a greater height (See FIG. 4), such as is used in robotic handling applications as it is the portion of the plate that is handled by the robotic system and greater height is generally needed in such applications. The bottoms of the wells are closed off by a porous filter 15 (as shown in FIG. 3). In this embodiment, one sheet of filter 15 is used and closes off each well 9 of the plate 1.

The holder 3 has a structure that conforms and fits to the underside and lip 14 of the plate. Its outer dimensions are substantially the same as or slightly less than the inner dimension of the lip 14 of the plate. The holder has a substantially planar bottom surface 21 and a substantially planar upper surface 23. The upper portion 25 of the holder 3 is indented from the bottom portion 27 of the holder 3 by a step 29. The lip 14 of the plate 1 fits and rests upon the horizontal portion of the upper surface 23 adjacent the step 29. The upper portion 25 of the holder 3 has substantially the same surface area dimensions as that of the filter area of the plate 1. The step 29 of the holder 3 is dimensioned such that the bottom of the filter 15 either touches or is in close approximation with the upper portion 25 of the holder 3. Preferably, the filter 15 and upper portion 25 do not touch so as to avoid any injury to the filter or its contents by such contact. The space between the filter 15 and the upper portion 25 should be kept to a minimum to provide as much support for the filter as possible. Suitable distances are from about 0.001 inch to about 0.1 inch (0.00254 cm to about 0.254 cm).

The attachment devices 5 may be of any type commonly used in the industry such as nuts and bolts, preferably with the nut being formed as an integral part of the holder; clips such as spring metal clips or plastic clips, clamps such as spring clamps, C-clamps, and the like.

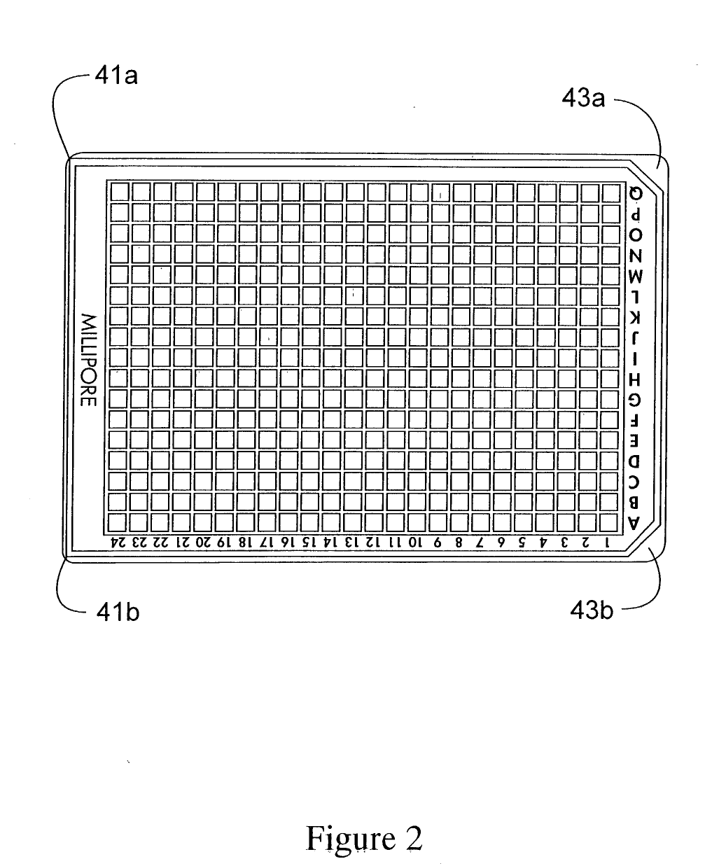

Alternatively, most plates are designed such that two corners of the plate along one edge are the same as each other and different from the other two corners (which are the same as each other) on the opposing edge of the plate. Typically two corners form a 90 degree angle. The other two corners on the opposing edge have the 90 degree corner clipped to form a 45 degree edge at the corner, see FIG. 2 wherein the first two corners 41A and B of plate 40 are at 90 degrees to the edges and the second two corners 43A and B are at 45 degrees to the intersecting edges. One can simply use this geometry to form a simple alignment/attachment device although something more robust and secure is typically desired.

As shown in FIGS. 1 and 3, the attachment devices are a series of metal spring clips, mounted on two or more sides 47 of the holder by one or more screws 49 per attachment device 45 (in this instance a clip).

FIG. 4 shows an embodiment of FIGS. 1-3 in which the lip 14A is of a height suitable for robotic handling. To the extent the reference numbers of FIG. 4 refer to the same component as is described in FIGS. 1-3 they remain the same. If they have been modified for this embodiment, they are followed by a letter A. As can be seen from FIG. 4 the lip 14A is of a greater height than the lip 14 of FIGS. 1 and 3 as is the step 29A and side 47A. Alternatively, the sides 47 may remain the same height as that of FIG. 3 and the attachment device 45 located lower on the lip 14A of the plate.

The plate may be any of the plates currently used or contemplated for use in gene sequencing or PCR product work. Such plates typically are formed of multiple wells, generally at least 96, preferably 384 and in some instances even 1536 wells. The plates are typically of standard size, such as 3.25 inches by 4.9 inches (8.26×12.45 cm). Each well has a depth to it that provides a defined volume for the sample. Typically these range from less than 0.1 microliters to about 200 microliters.

These plates are preferably formed of a thermoplastic material and are injection molded.

Suitable polymers which can be used to form the well plate include but are not limited to polycarbonates, polyesters, nylons, PTFE resins and other fluoropolymers, acrylic and methacrylic resins and copolymers, polysulphones, polyethersulphones, polyarylsulphones, polystyrenes, polyvinyl chlorides, chlorinated polyvinyl chlorides, ABS and its alloys and blends, polyolefins, preferably polyethylenes such as linear low density polyethylene, low density polyethylene, high density polyethylene, and ultrahigh molecular weight polyethylene and copolymers thereof, polypropylene and copolymers thereof, metallocene generated polyolefins and blends of polyolefins and other plastics such as PTFE resin. Preferred polymers are polyolefins, in particular polyethylenes and their copolymers, polystyrenes and polycarbonates. The polymers may be clear or rendered optically opaque or light impermeable. When using opaque or light impermeable polymers, it is preferred that their use be limited to the side walls so that one may use optical scanners or readers to read various characteristics of the retentate.

The bottoms of the wells are open and closed by a filter layer. The filter may be a single piece of filter that covers all of the wells of the plate with a single piece or it may be formed of two or more pieces covering one or more of the wells of the plate. In some instances, individual wells may be sealed by individual pieces of filter. Preferably, the filter is formed of one piece of material and attached to the bottom surface of the plate so as to close all of the wells. It is preferred that the filter be attached to the plate bottom so that it forms a liquid tight seal around the outer periphery of each well. Such devices are commercially available from Millipore Corporation of Bedford, Mass. in a variety of well configurations, filter choices and attachment techniques and are known by the brand name MULTISCREEN™ plates. Such plates and methods of making them and sealing their filters in place are also well known, see U.S. Ser. No. 09/844,304 and U.S. Ser. No. 09/305,696.

Suitable filters include microporous and ultrafiltration filters, typically in the form of porous filters or non-woven fabrics with ultrafiltration filters being preferred. The pore size for microporous membranes and filters may range from about 0.001 microns to about 10 microns, preferably from about 0.001 to about 1 micron, more preferably from about 0.001 to about 0.5 microns. Ultrafiltration membranes are described by the molecular cutoff of the molecule (often dissolved species of proteins and the like) that they retain. Typically, they have a molecular weight cutoff of from about 1 kiloDalton (kD) to about 30 kD, preferably from about 5 kD to about 15 kD.

Representative suitable microporous filters include nitrocellulose, cellulose acetate, polysulphones including polyethersulphone and polyarylsulphones, polyvinylidene fluoride, polyolefins such as ultrahigh molecular weight polyethylene, low density polyethylene and polypropylene, nylon and other polyamides, PTFE, thermoplastic fluorinated polymers such as poly (TFE-co-PFAVE), polycarbonates or particle-filled filters such as EMPORE® filters available from 3M of Minneapolis, Minn. Such filters are well known in the art and are commercially available from a variety of sources including Millipore Corporation of Bedford, Mass. If desired, these filters may have been treated to render them hydrophilic. Such techniques are well known and include but are not limited to grafting, crosslinking or simply polymerizing hydrophilic materials or coatings to the surfaces of the filters.

Representative ultrafiltration filters include polysulphones, including polyethersulphone and polyarylsulphones, polyvinylidene fluoride, and cellulose. These filters typically include a support layer that is generally formed of a highly porous structure. Typical materials for these support layers include various non-woven materials such as spun bounded polyethylene or polypropylene, or glass or microporous materials formed of the same or different polymer as the membrane itself. Such filters are well known in the art, and are commercially available from a variety of sources such as Millipore Corporation of Bedford, Mass.

The holder may be formed of any material that is capable of being formed into the desired shape and which provides suitable rigidity so as to support the filter even when under a downwardly directed pressure. Preferred materials include but are not limited to metals, such as stainless steel, aluminum, preferably anodized aluminum, copper, brass, bronze and white metal; plastics such as polycarbonates, polyesters, nylons, PTFE resins and other fluoropolymers, acrylic and methacrylic resins and copolymers, polysulphones, polyethersulphones, polyarylsulphones, polystyrenes, polyvinyl chlorides, chlorinated polyvinyl chlorides, ABS and its alloys and blends, polyolefins, preferably polyethylenes such as linear low density polyethylene, low density polyethylene, high density polyethylene, and ultrahigh molecular weight polyethylene and copolymers thereof, polypropylene and copolymers thereof, metallocene generated polyolefins and blends of polyolefins and other plastics such as PTFE resin and various thermoset materials such as epoxies, glass filled epoxies, phenolics and glass filled phenolics. Wood could also be used, however due to its absorptive properties it is not preferred.

The present invention is used in the following manner. A holder is formed that matches the dimensions of the desired plate (as described above) so as to provide a support for the filter. The plate, typically a 384 well plate such as MULTISCREEN™ plate is then processed using various liquids and other components to generate the desired reaction or purification product(s) in the wells. The plate 1 is then attached to the holder 3 as shown in FIG. 1 and secured to the holder 3 by the attachment devices 5. The samples in the wells are resuspended by the additional of one or more liquids, such as buffer solution or water to the wells that passively resuspend the samples. In cases where the samples will not passively resuspend, the samples after addition of the liquid(s) may be agitated such as by shaking or by drawing the liquid into and out of a pipette several times. The upper surface 7 of the plate 1 is then sealed with a piercable plenum (not shown) such as aluminum foil or silicone rubber and placed with the machine such as a DNA sequencer. The machine then performs a series of steps which either directly inject the sample of a well into an electrophoretic capillary or which inserts a pipette tip into the well and withdraws a specimen of the that sample and then injects it into an electrophoretic capillary. The method of drawing the sample is for a robotic arm to push a pipette tip through the piercable cover, or by lowering ends of the capillaries into the samples. One example of a well-known sequencer is the ABI 3700 DNA Sequencer that uses pressure resistance to determine the depth to which to insert the pipette tip. The support surface of the holder below the filter provides the necessary support to the filter to keep it from flexing so that the correct resistance is sensed and the proper sample obtained. Without the support layer below the filter, the filter would flex too much in a downward vertical motion, causing the pressure sensor of the robot to not sense the desired pressure feedback and to continue the advance of the tip or capillary into the well. This will result in either an error signal being generated in the machine or in some instances the piercing of the filter layer with the tip or capillary resulting in a loss of the specimen from that well.

In some applications, an incubation step or thermal cycling profile may be used as part of the sample preparation process. The holder itself may be thermally conductive so that it can supply the thermal energy to the filter and sample within the well or alternatively a thermally conductive layer can be applied to the top surface of the support layer to provide the thermal transfer. For example, the holder may be made of metal, such as stainless steel or a thermally conductive plastic such as graphite or metal filled plastic and it may be heated by a resistance heater, Peltier device, infrared or other radiant heat, steam and the like to suitable temperature. The holder then transmits the heat to the plate that is attached to it.

Alternatively, if the holder is made of non-thermally conductive material one may apply a thermally conductive layer to the upper support surface, such as metal coating or film, and apply the thermal energy to that coating or film in order to provide the heat to the plate. Examples of such a coating or film include but are not limited to stainless steel, copper or aluminum foil which is adhered or molded on to the surface of the plate and silver or graphite coatings, in a resin such as an epoxy, applied to the surface of the holder by spraying or roll coating.

A further alternative would be to use a rubber sheet containing one or more resistance heaters and interposing it between the plate and the upper support surface of the holder.

An unanticipated advantage of this invention is a simplified sample preparation process. If the samples are to be transferred immediately to a solid bottom plate for loading on the sequencer, resuspension is normally conducted by shaking or pipetting up and down. However, it has been found by the inventor that the samples will resuspend passively from the surface of the filter within the time (45 minutes) it takes for most commercial sequencers to complete the required pre-run of the instrument. When combined with the direct-inject holder this has the unanticipated benefit of eliminating time-consuming sample processing steps upstream.

Another advantage is the elimination of relatively expensive thermal cycling plates and other consumables. If both thermal cycling and direct-inject holder embodiments are utilized, two thermal cycling plates and hundreds of disposable pipette tips will be eliminated.

What is claimed:

1) A system formed of a multiple welled plate wherein each of the wells has an open bottom closed by a filter, a holder for such a plate wherein the holder is formed with a filter support portion that corresponds to at least a portion of the area of the filter contained in the plate and which supports at least that portion of a lower surface of the filter and one or more attachment devices for releasably holding the plate to the holder.

2) A holder for multiple welled plates comprising an outer periphery that matches an outer periphery of the plate, a filter support portion inward of the outer periphery that substantially matches the area of filter of the plate and one or more attachment devices for releasably securing the plate to the holder in a defined location.

3) The system of claim 1 wherein the holder is capable of providing the transfer of thermal energy to the filter of the plate.

4) The holder of claim 2 wherein the holder is capable of providing the transfer of thermal energy to the filter of the plate.

5) The system of claim 1 wherein the holder is capable of providing the transfer of thermal energy to the filter of the plate wherein the holder is formed of thermally conductive material.

6) The system of claim 1 wherein the holder is capable of providing the transfer of thermal energy to the filter of the membrane further comprising a thermally conductive layer placed between the lower surface of the filter and the support portion of the holder and the thermally conductive layer has a supply of thermal energy supplied to it.

7) The holder of claim 2 wherein the holder is capable of providing the transfer of thermal energy to the filter of the plate wherein the holder is formed of thermally conductive material.

8) The holder of claim 2 wherein the holder is capable of providing the transfer of thermal energy to the filter of the membrane further comprising a thermally conductive layer placed between the lower surface of the filter and the support portion of the holder and the thermally conductive layer has a supply of thermal energy supplied to it.

9) The system of claim 1 wherein the holder is electrically conductive.

10) The system of claim 1 wherein the liquid contained by the filter plate can be heated using thermally conductive pins from the open side of the plate.

11) The system of claim 10 wherein the thermally conductive pins are also electrically conductive.

12) The holder of claim 2 wherein the surface of the holder, or a shim, is at a distance between 0 and 2.5 mm from the filter bottom.

13) The holder of claim 2 wherein the filter plate can only be positioned in one orientation.

14) The holder of claim 2 wherein a part of the holder provides a means of actuating the DNA sequencer.

15) The holder of claim 1 wherein the holder presents the filter plate to the sequencer in a position that allows the samples to be transferred to the capillaries for injection.

16) A process for the direct injection processing comprising the steps of taking a multiple welled filter plate and preparing a series of purified samples in one or more of the wells, attaching the plate to a holder, the holder having a upper portion corresponding in size and location to the filter area of the plate so as to support the filter and limit its movement during processing, adding one or more fluids to the samples in the one or more wells to resuspend the samples, placing the plate/holder into a direct inject processing device and conducting a direct inject process to the samples in the one or more wells.

17) The system of claim 1 wherein the filter support portion corresponds substantially to the area of the filter.

18) The system of claim 1 wherein the filter support portion corresponds to two or more areas of the filter.

19) The system of claim 1 wherein the filter support portion is spaced apart from the lower surface of the filter.

20) The system of claim 1 wherein the filter support portion is spaced apart from the lower surface of the filter at a distance between 0 and 2.5 mm from the lower surface.
